(19) United States
(12) Patent Application Publication
(10) Pub. No.: US 2016/0324148 A1
(43) Pub. Date: Nov. 10, 2016

(54) HYDROGEL FORMULATION WITH MILD ADHESION

(71) Applicant: BIOCURE, INC., NORCROSS, GA (US)

(72) Inventors: Bruktawit Asfaw, Mableton, GA (US); Sarah Barbara Kokotoff, Dunwoody, GA (US); Fiona Patricia Clements, Stone Mountain, GA (US); Claude-Raymond René, Atlanta, GA (US); Dominic Dominikovich Ugbedah, Lawrenceville, GA (US); Christopher A. Zook, Kalamazoo, MI (US); Michael T. Sweeney, Portage, MI (US); Rebecca R. Quesnell, Kalamazoo, MI (US)

(21) Appl. No.: 15/147,657

(22) Filed: May 5, 2016

Related U.S. Application Data
(60) Provisional application No. 62/157,640, filed on May 6, 2015.

(51) Int. Cl. A01N 25/10; A61K 31/765; A61K 9/00

(52) U.S. Cl. CPC A01N 25/10; A61K 9/0041; A61K 31/765

(57) ABSTRACT

The present invention is generally in the field of methods, compositions, and devices for reducing the incidence of mastitis in an animal. More specifically the invention includes methods and compositions for creating a physical barrier on the teat surface or in the teat canal of an animal for prophylactic treatment of mammary disorders. In one embodiment, the methods and compositions are designed for use during the animal's dry period. In one embodiment, the methods and compositions are designed for placement in the teat cistern. In one embodiment, the methods and compositions are designed for placement in adhering to the external teat.

### FIELD OF THE INVENTION

The present invention is generally in the field of compositions having mild adhesion and medical and health related uses of such compositions. The invention is also in the field of methods, compositions, and devices for protecting the udder from pathogenic load and the resultant decrease in the incidence of mastitis in an animal. In one embodiment, the invention is methods, compositions, and devices for creating a physical barrier on the teat and udder surface or in the teat canal of an animal for prevention and/or treatment of mammary disorders. In one embodiment, the methods and compositions are designed for use in mid- to late-gestation and during the animal's dry period. In one embodiment, the methods and compositions are designed for placement in the teat cistern. In one embodiment, the methods and compositions are designed for placement in adhering to the external teat.

### BACKGROUND OF THE INVENTION

Mastitis is an inflammation of the mammary gland that is caused by bacteria which in most cases enter the gland via the teat orifice. During the non-lactating period or “dry period” in the gland, deposits of keratin in the teat orifice and the streak canal form a primary defense mechanism (schematic shown in FIG. 1). A keratin plug that forms in the teat of the animal forms a protective barrier, and the immune-rich tissues of the Furstenbürg's Rosette in the teat, as well as the natural protective factors of the dry-cow secretions, contain high levels of naturally occurring anti-bacterial substances which inhibit the passage of bacteria from the teat orifice to the teat cistern (papillary sinus) and gland cistern. However, this keratin plug and these natural immune defense mechanisms are often overcome by bacterial invasion as the animal is transitioning to the dry period, during the dry period of the animal, and/or during calving. This can result in bacteria invading the gland and causing mastitis during the dry period or, more particularly, immediately following calving.

Products have been developed to seal an animal's teat to prevent mastitis and other conditions. Barrier teat dips are designed for extra-mammary use, to seal the external surface and streak canal of the teat, and are typically used during periods of milking. For heifers, external dip products exist, but none sufficiently seal the teat of the cow externally for a sufficient amount of time to prevent the unique form of mastitis that is so dangerous in heifers. In some heifers and in cows that have experienced one or more pregnancies previously, teat sealants are also used to block or seal the teat canal during the dry period. Furthermore teat sealants are used to plug the teat cistern during the dry period.

U.S. Pat. Nos. 6,395,289 and 6,203,812 to Hydromer teach hydrophilic polymeric blends for use as teat dips during the lactating period. The blends provide barrier properties but can be rapidly removed prior to milking. However, the physical consistency and properties of these teat dips make them unsuitable for teat canal or cistern placement. For example, since these dips do not gel readily, they would tend to not remain in the canal or cistern.

Other teat dip compositions used during an animal's lactating period are disclosed in U.S. Pat. Nos. 4,113,854 and 5,017,369. Applied externally, these compositions form thick films which seal off the end of a teat canal. These compositions include latex and remain viscous and sticky thereby not allowing for teat canal protection from the environmental factors to which the gland is naturally exposed due to its location on the animal. Also, latex may be toxic or irritating to the mammary gland tissues. In addition to the contamination of milk, latex can elicit allergic reactions in humans.

U.S. Pat. No. 5,583,163 to Ciba Geigy and U.S. Pat. No. 5,932,674 to Novartis AG describe methods for the preparation of certain polyvinyl alcohol (PVA) polymers and hydrogels. U.S. Pat. No. 6,652,883 to BioCure teaches the use of PVA based hydrogels as bulking and sealing products. However, the formulations taught in the '883 patent do not have appropriate properties to be useful as intra-canal or intra-teat cistern sealants.

What is needed is a safe formulation that is easy and safe to apply and that preferably forms in place on the tissue. The formed hydrogel has mild adhesion, in the range of 0.05 to 0.5N.

### SUMMARY OF THE INVENTION

In one aspect, the invention is a hydrogel composition suitable for use, for example, as an animal teat sealant formed from macromers. The macromers can be in situ polymerized into the hydrogel teat sealant directly on or in an animal teat. In a preferred embodiment, the hydrogel composition is formed from a two part sterile liquid composition; one or both of the parts can contain the macromer, which are delivered to the teat in either a spray, dip, stream, or infusion manner, whereupon they combine and polymerize immediately to form the hydrogel. Optionally, active agents such as antimicrobial agents, analgesics, or anti-inflammatories may be included.

By rapidly forming the hydrogel when applied, the method of forming a teat sealant is fast, clean, “touchless” (hands free), and simple. Because it forms in situ, the sealant is highly conformal to the teat interior or exterior surface which ensures a better seal against infection and that any included active agents are more efficiently delivered directly to the teat.

The macromer is a water soluble synthetic polymer made by functionalizing a water soluble polyvinyl alcohol (PVA). PVA macromer in water may be unstable under certain sterilization and storage conditions. The composition thus may include means to stabilize the compositions prior to application. The PVA macromer is optimized by molecular weight, acetate content, and functional group content. These factors significantly affect viscosity, water uptake ability, hydrogel forming rate, adhesion, and mechanical properties of the hydrogel. A pure PVA hydrogel tends to dry out over a few hours, and this drying leads to a significant shrinkage and property changes of the hydrogel. Therefore, the composition may also contain moisturizers. In addition, thickening agents or density modifiers may be added to provide weight to the hydrogel to facilitate ease of use.

The macromers can be crosslinked through any appropriate means (such as application of ultraviolet light) but are preferably crosslinked using a H₂O₂/Fe(II) redox free radical initiation system. The reducing agent and oxidizing agent are separately packaged in the two composition parts, either or both of which can contain macromer.

The hydrogel has the qualities necessary to serve for many medical-related functions, including as a teat sealant. In particular the hydrogel has appropriate adhesion, swelling, and the mechanical strength allowing it to stay in place for an indefinite period of time.

In another embodiment, the teat sealant is a preformed injectable hydrogel.

In another embodiment, the teat sealant hydrogel forms an external cover on the teat.

### BRIEF DESCRIPTION OF THE DRAWINGS

FIG. 1 is a schematic of an animal teat.

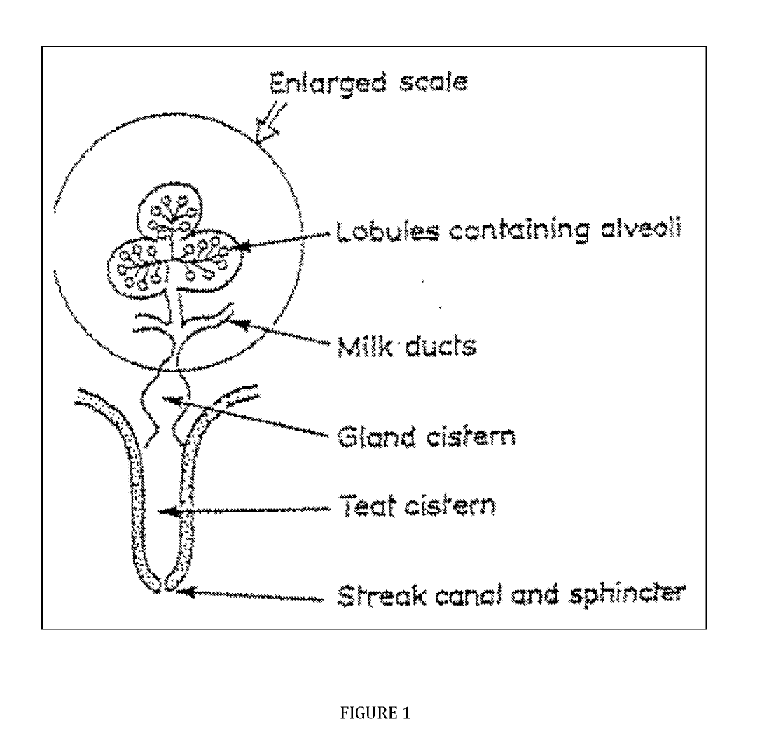

FIG. 2 is a chart of adhesion testing results.

FIG. 3 illustrates percent swelling of various formulations.

### DETAILED DESCRIPTION OF THE PREFERRED EMBODIMENTS

Compositions useful for forming hydrogel teat sealants, among other applications, are disclosed. The compositions include a macromolecular monomer (termed herein a “macromer”) that forms a hydrogel. The hydrogel is preferably formed in situ on or in the teat using a free radical initiation system or redox reaction.

In a preferred embodiment, the hydrogel is formed from macromers that are polymerized using a redox system. The reducing component includes the macromer and a reducing agent, with optionally a stabilizer and other additives. The oxidizing component includes the macromer and an oxidizing agent, with optionally a stabilizer and other additives. Both components are solutions.

The two component formulation is applied to the teat by a spray or stream from a syringe, pump, spray nozzle, aerosol, dip, or other type of device. The two components are desirably mixed through a static mixer and delivered to the teat. A combination of the spray and stream may be applied in a method similar to a shower head, whereby multiple streams provide the simulated broad coverage of a spray application. The macromers and other additives are sprayed or streamed to the teat whereupon they crosslink in situ to form the hydrogel-based teat sealant. For application inside the teat, as in the teat cistern, a syringe may be used.

The composition may further include one or more pharmaceutical agents, such as antimicrobial agents. The pharmaceutical agent or agents will become trapped in the hydrogel upon its formation and will be released from the hydrogel immediately or over a period of time.

In one embodiment the invention is a method and composition for forming a hydrogel physical barrier on a tissue surface, preferably on the surface of an animal teat.

In another embodiment the invention is a method and composition for forming a hydrogel physical barrier in a tissue, preferably in the teat cistern of an animal. The barrier may also be in the streak canal.

### DEFINITIONS

The term “teat sealant” as used herein refers to compositions and devices used to form a physical barrier on the surface of or inside an animal teat. A teat sealant can be on the teat surface, inside the teat streak canal, and/or inside the teat cistern.

The term “hydrogel” as used herein refers to a material having an aqueous phase with an interlaced polymeric component, with at least 10% and up to 95% of its weight as water.

The term “antimicrobial” as used herein refers to a substance that kills or inhibits the growth or reproduction of microorganisms such as bacteria, fungi, yeast, or protozoans.

The term “solution” as used herein refers to solutions, suspensions, or dispersions, unless otherwise stated.

The term “spray” as used herein refers to an atomized composition, such as comprised of small or large liquid droplets, such as applied through an aerosol applicator or pump spray applicator for the intended purpose of delivering a broad application of the composition.

The term “stream” as used herein refers to a continuous, direct, and focused application of the composition.

The term “infusion” as used herein refers to the continuous introduction of a fluid or solution into a cavity, vein or cistern.

The term “animal” as used herein refers to any female mammal which has a lactation period. The term “animal” preferably includes livestock animals, such as cows.

The term “heifer” as used herein refers to any young female cow that has not given birth to a calf.

The term “dry period” as used herein refers to the non-lactating phase of the lactation cycle of a cow. It occurs between the end of one lactation and the beginning of the next lactation.

The term “transition period” as used herein refers to the period before and after calving that includes the physiological, metabolic and endocrine changes associated with cessation of milk production for the non-lactating period (dry period) of the lactation cycle in a cow, including also the changes associated with the preparation for calving, preparation for milk production for the calf, calving and the period immediately following calving.

Formulation Characteristics

This patent covers formulations that have a unique combination of deliverability, swelling, and adhesion. More specifically, these formulations have a PVA concentration by weight of about 2% to about 10% (preferred range of about 4.5% to about 9%) and a glycerol concentration range of about 5 to 16% (preferred range of about 10% to about 16%). For teat sealants the range of glycerol content is about 5 to about 20%, preferably about 10 to about 16%. Characteristics of the hydrogel are a swelling range of about 100% to about 1000% of original weight (preferred range of about 400% to about 650%), and an Adhesion Test value of about 0.05 to about 0.5N, preferably about 0.35 to about 0.5N. For teat sealants in particular, the formulation has an Adhesion Test value of about 0.05 to about 0.35N, and more preferably about 0.07 to about 0.35N.

Teat Sealants

It should be understood that desired characteristics of a teat sealant will vary depending upon the intended usage of the sealant, such as where it will be applied, and other factors. However, some general characteristics can be stated.

The teat sealant formed is desirably easily removed from the teat. Where the sealant is placed intra-teat in the canal, it is desirably removed by stripping, most desirably in the first strip. In addition, the sealant is preferably removed as a single piece, or as multiple large pieces.

The in situ formed teat sealant should be conformable and compliant so that it conforms to the topography of the teat surface or canal and the tissue surface around the teat and is non-irritating to the animal. Conformability will also extend the longevity of the teat sealant. The teat sealant is also preferably strong enough that it can be peeled off the teat and can be removed in one cohesive unit leaving little material behind. Thus, it is often desirable to control the adhesion of the teat sealant. This can be achieved by modifying the macromer with hydrophilic or hydrophobic side chains, including an additional monomer, or adding specific additives such as surfactants, organic solvents, or amphiphilic block copolymers (e.g. pluronics).

Hydrogels that have a specific combination of adhesion and swelling will exhibit properties that are novel in the creation of removable barriers compatible with human or animal tissue. These hydrogels can be used as plugs at the macro or micro level, and the appropriate range of adhesion will allow these in situ formed hydrogels to adhere to tissue and stay in place all while being easily removed manually. In addition, if these hydrogels are plugged in a location where a barrier to retain/absorb fluid is necessary, the hydrogels can naturally absorb the fluid while remaining in their desired location. Preferred values for adhesion range from about 0.05 to about 0.50N. Preferred values for swelling are from about 100% to about 1000% of original weight.

The hydrogel teat sealant additionally needs to be safe and stable. All of the composition ingredients should be biocompatible or non-irritant in the amounts present in the final hydrogel teat sealant. The composition should be sterile and able to preserve the activity of any optionally included drug.

The viscosity of the composition should be suitable for the delivery method. The viscosity should be controlled so that the composition can be sprayed or streamed onto or into the teat in a way that a conformal teat sealant is generated. Viscosity can be controlled by changing the molecular weight and concentration of the macromer.

Gelation of the macromer composition on the teat is preferably rapid, to avoid run off of the composition from the place of application. The gelling time can be about 5 minutes or less, preferably less than about three minutes, more preferably less than about 0.5 minute, and, in some situations, as low as about 10 seconds or less. The gelling time is controlled by many factors, such as adjusting the concentration of initiators, type of initiator, crosslinking group of the PVA macromer, solids content of the composition, and mixing mechanism.

Generally about 0.5 to about 5 g of the composition will be administered to an animal teat, more preferably about 1.0 to about 4.0 g. Desirably the composition is administered during the transition period through the dry period of a heifer or cow. The composition can be administered through infusion into the teat or by external application to the teat.

Components of the Composition

The composition includes a PVA macromer that can quickly crosslink after delivery, to form a teat sealant in or on a teat. The composition further includes aqueous media, redox components, stabilizers for the redox components, thickening agents, and optionally one or more antimicrobial agents, and may include additives such as an absorbent, and other active agents.

PVA Macromer:

The macromer can be made by general synthetic methods known to those skilled in the art. The preferred macromers can be made as described in U.S. Pat. Nos. 5,508,317, 5,583,163, 5,583,163, 5,665,840, 5,807,927, 5,849,841, 5,932,674, 5,932,674, 5,939,489, and 6,011,077. The macromer has at least two pendant chains containing groups that can be crosslinked. The term “group” includes single polymerizable moieties containing vinyl groups such as an acrylate and acrylamide. The crosslinkers are desirably present in an amount of from about 0.01 to 10 millimole of crosslinker per gram of backbone (mmol/g), more desirably about 0.05 to 1.0 mmol/g. The macromer can contain more than one type of crosslinkable group. The pendant chains are attached via the hydroxyl groups of the backbone. Desirably, the pendant chains having crosslinkable groups are attached via cyclic acetal linkages to the 1,2-diol or 1,3-diol hydroxyl groups. Desirable crosslinkable groups include (meth)acrylamide, (meth)acrylate, styryl, vinyl ester, vinyl ketone, and vinyl ethers. Particularly desirable are ethylenically unsaturated functional groups.

A particularly desirable crosslinker is N-acryloyl-aminoacetaldehyde dimethylacetal (NAAADA) (CAS 49707-23-5) in an amount from about 1 to about 500 crosslinkers per macromer. A particularly preferred macromer has a PVA backbone (67 kDa, 12% acetate incorporation) modified with 0.1 mmol/g N-acrylamidoacetaldehyde dimethyl acetal (NAAADA) pendant polymerizable groups (PVA 888-7X) (Moxiol 8-88 (88% hydrolyzed) with 7 crosslinks per chain). Hydrophilicity of the PVA macromer can be adjusted by reacting with hydrophobic acetal or hydrophilic ammonium acetal. Macromers can also be modified to enhance the hydrogel tackiness, change the solution viscosity and gellation speed, and to change the hydrogel water content, absorption capability, and mechanical properties.

Crosslinking Initiators:

The macromers are polymerized by redox free radical polymerization using a two-part redox system. One part of the system contains a reducing agent such as ferrous salt. Various ferrous salts can be used, such as ferrous gluconate dihydrate, ferrous sulfate, ammonium ferrous sulfate, ferrous lactate dihydrate, or ferrous acetate. The amount of reducing agent used will vary. In one embodiment, the percent range for ferrous salt in the reductant component is about 0.06-0.18%. The other part of the composition contains an oxidizing agent such as hydrogen peroxide. The amount of oxidizing agent used will also vary. In one embodiment, the percent range for oxidizing agent in the oxidant component is about 0.05-0.12% grams in 10 mL. Either or both of the redox solutions can contain macromer. The agents react to initiate the polymerization of the macromer to generate a crosslinked hydrogel. Other reducing agents can be used, including but not limited to, iron, titanium trichloride, cysteine, and sodium thiosulfate. Other oxidizing agents that can be used include, but are not limited to, ammonium persulfate, ceric (IV) salt, and t-butyl hydroperoxide.

Stabilizers:

The oxidizing component is stabilized by peroxide stabilizers such as sodium pyrophosphate or organophosphonates (Dequest® 2010 and Dequest® 2060S, Solutia Inc.). Phosphonates are chelants that offer stabilization of peroxide systems. Dequest® 2010 is 1-hydroxy ethylene-1,1-diphosphonic acid. The active in Dequest® 2060S is diethylenetriamine penta(methylene phosphonic acid). Other chelating agents are EDTA and citric acid. These can be added in amounts as recommended by the manufacturer, generally less than 200 ppm. The reducing component is stabilized using antioxidant stabilizers, including but not limited to ascorbic acid (vitamin C), glutathione, lipoic acid, uric acid, carotenes (vitamin A), BHT, BHA, propyl gallate, and α-tocopherol (vitamin E). Generally the antioxidant stabilizer is added at below 1% by weight. Antioxidants also function to stabilize the macromers by inhibiting polymerization.

Antimicrobial Active Agents:

There are a variety of antibacterial agents available for use in animals. These antibacterial agents include, but are not limited to, the following: macrolides, for example, tulathromycin (Draxxin®), tildipirosin (Zuprevo®), tilmicosin (Micotil®), tylosin phosphate (Tylan®), and gamithromycin (Zactran®); cephalosporins, for example, ceftiofur sodium (e.g., Naxcel® and Excenel®), ceftiofur hydrochloride (e.g., Excenel RTU Excenel RTU EZ®, Spectramast®), ceftiofur crystalline free acid (Excede®), cefovecin sodium (Convenia®), and cefpodoxime proxetil)(Simplicef®); lincosaminide antibiotics, for example, lincomycin (Lincomix®), pirlimycin hydrochloride (Pirsue®), and clindamycin hydrochloride (Antirobe®); fluoroquinolones, for example, danofloxacin (Advocin®), enrofloxacin (Baytril), and marbofloxacin (Zeniquin®); and tetracyclines, for example, chlortetracycline, oxytetracycline, and doxycycline. Other antibacterial agents include, but are not limited to, amoxicillin trihydrate and clavulonic acid (Clavamox®), spectinomycin (Adspec®), potentiated sulfonamides including trimethoprim/sulfadiazine (Tucoprim®) and sulfadimethoxine/ormetoprim (Primor®); and florfenicol (for example, Nuflor® and Nuflor® Gold). An antimicrobial or pharmaceutical agent may be administered simultaneously or sequentially with the compositions of the present invention.

Density Modifiers/Thickening Agents

Agents may be added to provide weight to the hydrogel and prevent the hydrogel from migrating up the teat cistern and into the gland cistern during the dry period. Examples of appropriate density modifiers or thickening agents include but are not limited to the following: partially and fully hydrolyzed polyvinyl alcohol, dextrin, distarch phosphate, edible gelatin, polydextroses, propylene glycol, methyl cellulose, oxidized starch, ammonium dihydrogen phosphate, Glucoamylase, Calcium dihydrogen phosphate, Calcium malate (DL1), Calcium monohydrogen phosphate, Carotene, L-glutamic acid, Magnesium hydrogen phosphate, Mallitol, Manitol, Disodium dihydrogen phosphate, Starch acetate, Polydextroses, Processed Eucheuma seaweed, Propylene glycol, Gellan gum, Edible gelatin, Carrageenan, Sorbitol, Methyl cellulose, Disodium ethylenediaminetetraacetate, Calcium disodium ethylenediaminetetraacetate, Potassium dihydrogen citrate, Potassium dihydrogen phosphate, Oxystearin, Hydroxypropyl distarch phosphate, Hydroxypropyl methyl cellulose, Tara Gum, Tragacanth Gum, Xanthan Gum, Guar Gum, Gum Arabic and Potassium alginate.

The thickener can be added at about 1.5 to about 10% by weight of the composition. The final density of the formulation may be about 1.1 to about 2.0 g/cc.

Other Components:

The composition may additionally contain one or more additives such as stabilizers, defoamers, pore forming agents, plasticizers, penetration enhancers, colorants, wettings agents, leveling agents, thickeners, fillers, opacifying agents, and absorbents.

Other additives can include pain relief agents, analgesics, and/or anti-inflammatories such as, but not limited to: lidocaine, ibuprofen, diclofenac, and capsaicin.

The composition may contain various additives including but not limited to glycerol, polyethylene glycol, polypropyl glycol, polybutylene glycol, polyacrylic acid, celluloses, calcium alginate, sucrose, lactose, fructose, sorbitol, mannitol, zylitol, dextrans, hyaluronic acid, polyacrylamidopropyltrimethyl ammonium chloride, calcium chloride, APOSS (Octaammonium-POSS (polyhedral oligomeric silsesquioxane)), and poly(2-acrylamido-2-methylpropane sulfonic acid). These can be added to the composition to improve the performance of the teat sealant including adhesion, tackiness, and to change the water content, water uptake, and moisture vapor transmission (MVTR).

The composition is steam sterilizable and can be stored or packaged under vacuum or an inert atmosphere of nitrogen or argon in order to prevent oxidation of the reductant initiator component.

Adhesion Control Using Glycerol

The above described beneficial characteristics for a teat sealant such as adhesion, swelling, tack, moisture handling, fluid absorption, and viscosity can be achieved with certain formulations. In particular, it has been found that about 5 to about 16% by weight glycerol provides the desired degree of adhesion and other qualities for a hydrogel teat sealant formed from PVA based macromers.

Delivery of the Compositions

Appropriate viscosity depends upon the delivery means to be employed. Generally, the composition should have a viscosity lower than about 800 cps, preferably lower than about 300 cps, more preferably lower than about 200 cps to be delivered via aerosol. Delivery through a pump spray generally requires a lower viscosity, such as less than about 150 cps. Spray without aerosol calls for a viscosity less than about 50 cps.

The composition is delivered to the teat from a spray device or a stream device. The spray device includes a container having a dispenser for spray delivery of the liquid composition. The type of container used is variable, depending upon compatibility with the composition and the spray dispenser and can be glass, plastic, or metal. If the solutions are of a low enough viscosity, a spray delivery may be achieved with simple mechanical forces such as those achieved when depressing the plunger of a syringe by hand through an appropriately designed nozzle.

The composition can also be delivered using a syringe outfitted with a spray head, or a dual spray device outfitted with a spray head and, optionally, a mixing chamber. Generally, any chemical, mechanical or electronic method for propelling the liquid composition as a spray from the container is appropriate. In one embodiment, a compatible liquid or gaseous aerosol propellant is placed in an appropriate container along with the composition and the dispenser includes a valve mechanism that enables atomized spray delivery of the liquid composition.

A device is used having two containers so that the components are kept apart until used. The device can have a single dispenser, such as a spray tip from Nordson Corp. or a device having a double dispenser, e.g. a bar spray tip from Micromedics can be used. If a double dispenser is used, the sprays from the dispensers can be aligned to substantially overlap. A suitable device is described in U.S. Pat. No. 5,989,215, for example. It is also possible, although less preferred, to apply the two solutions sequentially. A mixer may be employed in the case of a single dispenser to mix the two solutions before or during spraying. The device may include a meter so that the quantity of composition can be controlled.

The composition is applied to the teat as a stream or spray using an appropriate delivery device. The composition should be applied to result in a hydrogel having a thickness ranging from about 0.01 to about 5 mm, desirably about 0.1 to about 3 mm. It may be desirable to apply several layers of the composition to the teat to ensure adequate coverage of the teat.

Examples of devices that could be used, or modified for use, to deliver the compositions include those described in WO 2015/038281 to Zoetis, US 2015/0080841 to Zoetis LLC, U.S. Pat. No. 5,989,215 to Baxter International Inc., U.S. Pat. No. 8,353,877 to Bimeda Research & Development, WO 2003/022245 to Bimeda Research & Development, and WO 2013/021186 to Norbrook Laboratories Limited.

### EXAMPLES

The examples below serve to further illustrate the invention, to provide those of ordinary skill in the art with a complete disclosure and description of how the compounds, compositions, articles, devices, and/or methods claimed herein are made and evaluated, and are not intended to limit the scope of the invention. In the examples, unless expressly stated otherwise, amounts and percentages are by weight, temperature is in degrees Celsius or is at ambient temperature, and pressure is at or near atmospheric. The examples are not intended to restrict the scope of the invention.

Modifications and variations of the present invention will be apparent to those skilled in the art from the forgoing detailed description. All modifications and variations are intended to be encompassed by the following claims. All publications, patents, and patent applications cited herein are hereby incorporated by reference in their entirety.

### Example 1

### Formulations

Hydrogel formulations were prepared from two solutions, an oxidant solution and a reductant solution, which were combined to form the hydrogel. The two solutions contained some ingredients in equal parts (macromer and glycerol) but only the oxidant solution contained hydrogen peroxide and only the reductant solution contained Fe(II), ascorbic acid, and calcium chloride.

The components of the oxidant were mixed into a homogeneous solution. Once fully mixed, the oxidant component was dispensed into one part of a dual barrel syringe. The components of the reductant were similarly mixed into a homogenous solution. After thorough mixing, the reductant component was dispensed into the second part of the dual barrel syringe.

The macromers used in the formulations were PVA of the molecular weights noted, substituted with the noted amounts of N-acrylamidoacetaldehyde dimethyl acetal (NAAADA). The macromers were prepared applying the method of Example 15 from U.S. Pat. No. 5,932,674, and were isolated as aqueous 200 mg/mL solutions for formulation.

Formulation A
Formulation A - Oxidant solution

| Formulation | | Final Composition | |
|---|---|---|---|
| Component | Weight % | Ingredient | mg/g |
| Macromer 1 solution^{a} | 31.0 | Macromer 1 | 60 |
| H₂O₂ solution^{c} | 1.5 | H₂O₂ | 0.8 |
| PVA 3-83 solution^{b} | 0.0 | PVA 3-83 | 0 |
| Chelant solution^{d} | 8.1 | Diethylenetriamine | 0.1 |
| | | penta(methylene | |
| | | phosphonic acid) | |
| Glycerol | 16.0 | Glycerol | 160 |
| Water | 22.7 | Water | qs 1 g |

Formulation A - Reductant solution

| Formulation | | Final Composition | |
|---|---|---|---|
| Component | Weight % | Ingredient | mg/g |
| Macromer 1 solution^{a} | 31.0 | Macromer 1 | 60 |
| Iron/ascorbic acid solution^{e} | 13.8 | Ferrous lactate | 1.6 |
| | | Ascorbic acid | 2.2 |
| Calcium chloride | 10.7 | Calcium chloride | 107 |
| Glycerol | 16.0 | Glycerol | 160 |
| Water | 16.7 | Water | qs 1 g |

^{a}Macromer I solution: aqueous 200 mg/mL Macromer 1 (Macromer 1: 67 kDa PVA functionalized with 0.1 mmol/g NAAADA)
^{b}PVA Mowiol ® 3-83 solution: aqueous 300 mg/mL PVA 3-83
^{c}H₂O₂ solution: aqueous 50 mg/mL hydrogen peroxide
^{d}Chelant solution: 150 μL Dequest ® 2060S diluted in water to 100 mL
^{e}Aqueous 11 mg/mL ferrous lactate plus 15 mg/mL ascorbic acid

Formulation A4
Formulation A4 - Oxidant solution

| Formulation | | Final Composition | |
|---|---|---|---|
| Component | Weight % | Ingredient | mg/g |
| Macromer 1 solution^{a} | 31.0 | Macromer 1 | 60 |
| PVA 3-83 solution^{b} | 20.7 | PVA 3-83 | 60 |
| H₂O₂ solution^{c} | 1.5 | H₂O₂ | 0.8 |
| Chelant solution^{d} | 8.1 | Diethylenetriamine | 0.1 |
| | | penta(methylene | |
| | | phosphonic acid) | |
| Glycerol | 16.0 | Glycerol | 160 |
| Water | 22.7 | Water | qs 1 g |

Formulation A4 - Reductant solution

| Formulation | | Final Composition | |
|---|---|---|---|
| Component | Weight % | Ingredient | mg/g |
| Macromer 1 solution^{a} | 31.0 | Macromer 1 | 60 |
| PVA 3-83 solution^{b} | 13.3 | PVA 3-83 | 40 |
| Iron/ascorbic acid solution^{e} | 13.8 | Ferrous lactate | 1.6 |
| | | Ascorbic acid | 2.2 |
| Calcium chloride | 10.7 | Calcium chloride | 107 |
| Glycerol | 16.0 | Glycerol | 160 |
| Water | 16.7 | Water | qs 1 g |

^{a}Macromer I solution: aqueous 200 mg/mL Macromer 1 (Macromer 1: 67 kDa PVA functionalized with 0.1 mmol/g NAAADA)
^{b}PVA, Mowiol ® 3-83 solution: aqueous 300 mg/mL PVA 3-83
^{c}H₂O₂ solution: aqueous 50 mg/mL hydrogen peroxide
^{d}Chelant solution: 150 μL Dequest ® 2060S diluted in water to 100 mL
^{e}Aqueous 11 mg/mL ferrous lactate plus 15 mg/mL ascorbic acid

Formulation I
Formulation I - Oxidant solution

| Formulation | | Final Composition | |
|---|---|---|---|
| Component | Weight % | Ingredient | mg/g |
| Macromer 2 solution^{a} | 34.0 | Macromer 2 | 60 |
| PVA 3-83 solution^{b} | 0.0 | PVA 3-83 | 0 |
| H₂O₂ solution^{c} | 1.5 | H₂O₂ | 0.8 |
| Chelant solution^{d} | 8.3 | Diethylenetriamine | 0.1 |
| | | penta(methylene | |
| | | phosphonic acid) | |
| Glycerol | 16.0 | Glycerol | 160 |
| Water | 40.4 | Water | qs 1 g |

Formulation I - Reductant solution

| Formulation | | Final Composition | |
|---|---|---|---|
| Component | Weight % | Ingredient | mg/g |
| Macromer 2 solution^{a} | 34.0 | Macromer 2 | 60 |
| PVA 3-83 solution^{b} | 0.0 | PVA 3-83 | 0 |
| Iron/ascorbic acid solution^{e} | 1.6 | Ferrous lactate | 1.6 |
| | | Ascorbic acid | 2.2 |
| Calcium chloride | 10.7 | Calcium chloride | 107 |
| Glycerol | 16.0 | Glycerol | 160 |
| Water | 25.5 | Water | qs 1 g |

^{a}Macromer 2 solution: aqueous 200 mg/mL Macromer 2 (Macromer 2: 130 kDa PVA functionalized with 0.1 mmol/g NAAADA)
^{b}PVA Mowiol ® 3-83 solution: aqueous 300 mg/mL PVA 3-83
^{c}H₂O₂ solution: aqueous 50 mg/mL hydrogen peroxide
^{d}Chelant solution: 150 μL Dequest ® 2060S diluted in water to 100 mL
^{e}Aqueous 11 mg/mL ferrous lactate plus 15 mg/mL ascorbic acid

Formulation I4
Formulation I4 - Oxidant solution

| Formulation | | Final Composition | |
|---|---|---|---|
| Component | Weight % | Ingredient | mg/g |
| Macromer 2 solution^{a} | 26.7 | Macromer 2 | 47 |
| PVA 3-83 solution^{b} | 16.7 | PVA 3-83 | 50 |
| H₂O₂ solution^{c} | 1.5 | H₂O₂ | 0.8 |
| Chelant solution^{d} | 8.1 | Diethylenetriamine | 0.1 |
| | | penta(methylene | |
| | | phosphonic acid) | |
| Glycerol | 16.0 | Glycerol | 160 |
| Water | 31.1 | Water | qs 1 g |

Formulation I4 - Reductant solution

| Formulation | | Final Composition | |
|---|---|---|---|
| Component | Weight % | Ingredient | mg/g |
| Macromer 2 solution^{a} | 26.7 | Macromer 2 | 50 |
| PVA 3-83 solution^{b} | 16.7 | PVA 3-83 | 50 |
| Iron/ascorbic acid solution^{e} | 13.3 | Ferrous lactate | 1.6 |
| | | Ascorbic acid | 2.0 |
| Calcium chloride | 10.7 | Calcium chloride | 107 |
| Glycerol | 16.0 | Glycerol | 160 |
| Water | 16.7 | Water | qs 1 g |

^{a}Macromer 2 solution: aqueous 200 mg/mL Macromer 2 (Macromer 2: 130 kDa PVA functionalized with 0.1 mmol/g NAAADA)
^{b}PVA Mowiol ® 3-83 solution: aqueous 300 mg/mL PVA 3-83
^{c}H₂O₂ solution: aqueous 50 mg/mL hydrogen peroxide
^{d}Chelant solution: 150 μL Dequest ® 2060S diluted in water to 100 mL
^{e}Aqueous 11 mg/mL ferrous lactate plus 15 mg/mL ascorbic acid

### Example 2

### Swelling and Adhesion Testing

For adhesion testing, samples were conditioned at 37° C. for 2 hrs prior to testing. Adhesion data were generated between 0 to 2 hours after in situ formulation of the hydrogel. The swelling data is reported herein after 72 hours of swelling. At lesser times, the extent of swelling is less.

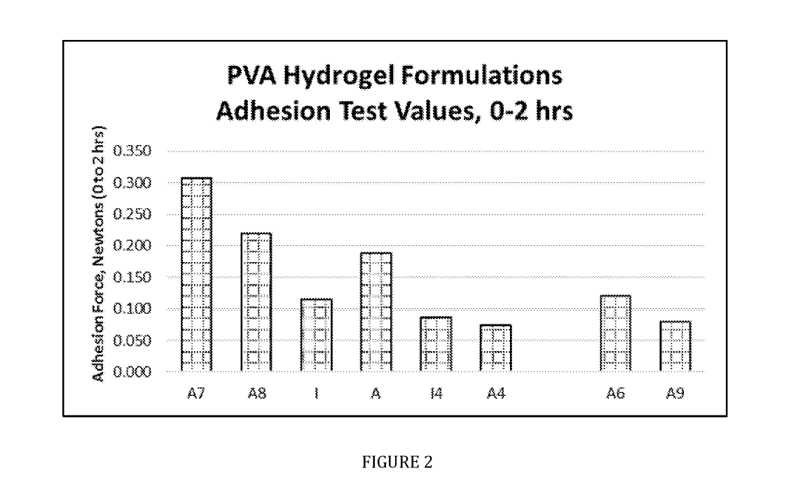

For adhesion testing (referred to herein as “Adhesion Test”), peel test samples were made using a 60 mm×50 mm×3 mm mold attached to damp (or wet) collagen with an affixed gauze backing to facilitate the peeling. To assess adhesion on these samples, a modified procedure from the standard 180° peel test was performed with the principal modification of setting the sample horizontally as opposed to vertically. A high-precision universal peel tester was used to measure the force to pull the backing “on itself”. The pulling was done at a constant speed, and a plot of force over time of peel was generated. The reporting force is the maximum recorded force during the pulling of the gauze+sample from the wet, affixed collagen backing. Adhesion Test results are shown in FIG. 2.

For the swell testing, cylindrical preformed gel samples were prepared by slowly administering the macromer to ensure no bubbles into a 6 cm×0.5 cm piece of rigid plastic tubing. The formed hydrogel was extracted from the tubing and samples used were between 1-2 g.

Test Method (Dehydration): Prepared samples were first dehydrated by placing in oven at 45° C. over a period of time until there was no significant weight change. Sample weights were taken at time 0 and then again every 24 hours until full dehydration is achieved.

Test Method (Swelling): Fully dehydrated samples were placed in sealed test tubes containing 15 mL PBS solution at 25° C., 60% RH and allowed to swell. Sample weight of the fully dehydrated sample is considered time 0 in this case. Sample weights were taken at time 0, 1 hr, 2 hr, 24 hr, 48 hr, 72 hr, and 7 days.

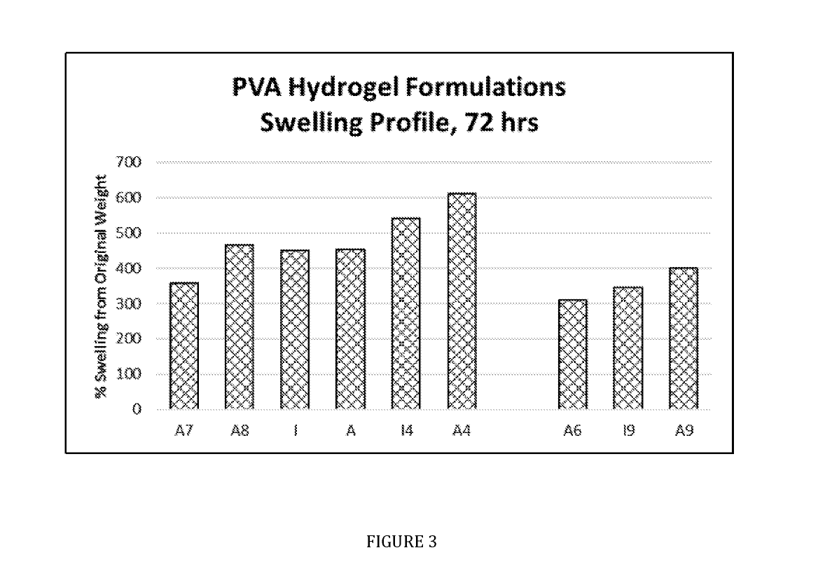

Adhesion and swelling testing results are shown in FIGS. 2 and 3, respectively. Referring to FIG. 2, samples A7, A8, I, A, I4, and A4 have PVA content between 4.5 and 9.0% by weight, and glycerol content between 5% and 16% by weight. Samples A7, A8, I, A, and 14 exhibit adhesion range from 0.07N to 0.35 N. Referring to FIG. 3, samples A8, I, A, I4, and A4 swelled 400 to 650% after 72 hours. The samples depicted in FIGS. 2 and 3 are summarized below:

| | PVA | % PVA | % Glycerol | Filler |
|---|---|---|---|---|
| Formulation | Mol. Wt. | (weight %) | (weight %) | (weight %) |
| A6 | 67 kDa | 6.00 | 0.0 | |
| I9 | 130 kDa | 6.02 | 23.0 | |
| A9 | 67 kDa | 6.03 | 25.0 | |
| A7 | 67 kDa | 6.03 | 5.0 | |
| A8 | 67 kDa | 6.03 | 10.0 | |
| I | 130 kDa | 6.02 | 16.0 | |
| A | 67 kDa | 6.03 | 16.0 | |
| I4 | 130 kDa | 4.73 | 16.0 | 5% of aqueous 30% |
| | | | | 3-83 Mowiol ® Filler¹ |
| A4 | 67 kDa | 6.03 | 16.0 | 5% of aqueous 30% |
| | | | | 3-83 Mowiol ® Filler¹ |

¹Mowiol ® 3-83 is a partially hydroyzed polyvinyl alcohol marketed by Clariant.

What is claimed is:

1. A hydrogel composition formed from:
a PVA macromer in an amount between about 2 and about 10 wt %; and
glycerol in an amount between about 5 and about 20 wt %;
and wherein the hydrogel has an Adhesion Test value of between about 0.05 to about 0.5N and a swelling range of between about 100% to about 1000% of original weight.

2. The hydrogel composition of claim 1, wherein the glycerol is present in an amount between about 10 to about 15 wt %.

3. The hydrogel composition of claim 1, wherein the glycerol is present in an amount between about 5 and about 15%.

4. The hydrogel composition of claim 1, wherein the glycerol is present in an amount between about 10 and 16%.

5. The hydrogel composition of claim 1, wherein the Adhesion Test value is between about 0.15 to 0.5N.

6. The hydrogel composition of claim 1, wherein the Adhesion Test value is between about 0.05 to 0.2N.

7. The hydrogel composition of claim 1, wherein the Adhesion Test value is between about 0.35 to 0.5N.

8. The hydrogel composition of claim 1, wherein the Adhesion Test value is between about 0.07 to 0.2N.

9. The hydrogel composition of claim 1, wherein the Adhesion Test value is between about 0.07 to 0.19N.

10. The hydrogel composition of claim 1, wherein the Adhesion Test value is between about 0.07-0.35 N.

11. The hydrogel composition of claim 1, wherein the PVA macromer is present in an amount between about 4.5-9 wt %.

12. The hydrogel composition of claim 1, further including an active agent.

13. The hydrogel composition of claim 8, wherein the active agent is a pharmaceutical agent such as an antimicrobial.

14. A hydrogel composition formed from:
a PVA macromer in an amount between about 4.5 and about 9 wt %; and
glycerol in an amount between about 10 and about 16 wt %;
and wherein the hydrogel has an Adhesion Test value of between about 0.07 to about 0.35N and a swelling range of between about 400% to about 650% of original weight.

15. The hydrogel composition of claim 14, wherein the PVA macromer is PVA functionalized with NAAADA.

16. The hydrogel composition of claim 15, wherein said PVA has a molecular weight of 67 kDa or 130 kDa.

17. The hydrogel composition of claim 14, wherein the PVA macromer is polymerized by redox free radical polymerization.

18. The hydrogel composition of claim 1, wherein the PVA macromer is PVA functionalized with NAAADA.

19. The hydrogel composition of claim 18, wherein said PVA has a molecular weight of 67 kDa or 130 kDa.

20. The hydrogel composition of claim 1, wherein the PVA macromer is polymerized by redox free radical polymerization.
